SEQUENCE LISTING (23 sequences)

SEQ ID NO: 1 - DNA, 99 bp, Artificial Sequence
  Synthetic Oligonucleotide
aaataatgattttattttgactgatagtgacctgttcgttgcaacaaattgatgagcaatgcttttttataatgccaactttgtacaaaaaagcaggct

SEQ ID NO: 2 - DNA, 100 bp, Artificial Sequence
  Synthetic Oligonucleotide
caaataatgattttattttgactgatagtgacctgttcgttgcaacaaattgataagcaatgctttcttataatgccaactttgtacaagaaagctgggt

SEQ ID NO: 3 - DNA, 35 bp, Artificial Sequence
  Synthetic Oligonucleotide
ttttataatgccaactttgtacaaaaaagcaggct

SEQ ID NO: 4 - DNA, 35 bp, Artificial Sequence
  Synthetic Oligonucleotide
tcttataatgccaactttgtacaagaaagctgggt

SEQ ID NO: 5 - DNA, 49 bp, Artificial Sequence
  Synthetic Oligonucleotide
gatgagcaatgcttttttataatgccaactttgtacaaaaaagcaggct

SEQ ID NO: 6 - DNA, 49 bp, Artificial Sequence
  Synthetic Oligonucleotide
gataagcaatgctttcttataatgccaactttgtacaagaaagctgggt

SEQ ID NO: 7 - DNA, 62 bp, Artificial Sequence
  Synthetic Oligonucleotide
gttgcaacaaattgatgagcaatgcttttttataatgccaactttgtacaaaaaagcaggct

SEQ ID NO: 8 - DNA, 62 bp, Artificial Sequence
  Synthetic Oligonucleotide
gttgcaacaaattgataagcaatgctttcttataatgccaactttgtacaagaaagctgggt

SEQ ID NO: 9 - DNA, 73 bp, Artificial Sequence
  Synthetic Oligonucleotide
gtgacctgttcgttgcaacaaattgatgagcaatgcttttttataatgccaactttgtacaaaaaagcaggct

SEQ ID NO: 10 - DNA, 73 bp, Artificial Sequence
  Synthetic Oligonucleotide
gtgacctgttcgttgcaacaaattgataagcaatgctttcttataatgccaactttgtacaagaaagctgggt

SEQ ID NO: 11 - DNA, 14 bp, Artificial Sequence
  Synthetic Oligonucleotide
cctgcaggaccatg

SEQ ID NO: 12 - DNA, 16 bp, Artificial Sequence
  Synthetic Oligonucleotide
cttaattaagaccatg

SEQ ID NO: 13 - DNA, 9 bp, Artificial Sequence
  Synthetic Oligonucleotide
ctcgagcta

SEQ ID NO: 14 - DNA, 17 bp, Artificial Sequence
  Synthetic Oligonucleotide
cctgcaggctcgatcta

SEQ ID NO: 15 - DNA, 1812 bp, Bacillus subtilis
agaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagttcctgagttcgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgcctagcttcctgctgaacatcaaaggcaagaaaacatctgttgtcaaagacagcatccttgaacaaggacaattaacagttaacaaataaaaacgcaaaagaaaatgccgatatcctattggcattga

SEQ ID NO: 16 - DNA, 306 bp, Bacillus subtilis
atgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataa

SEQ ID NO: 17 - DNA, 35 bp, Artificial Sequence
  Synthetic Oligonucleotide
ctcctgcaggaccatggtcagccatccaatggaga

SEQ ID NO: 18 - DNA, 35 bp, Artificial Sequence
  Synthetic Oligonucleotide
ctgggtctcgagctaatgaaatcccatagtagctt

SEQ ID NO: 19 - DNA, 67 bp, Artificial Sequence
  Synthetic Oligonucleotide
ccccgatgagcaatgcttttttataatgccaactttgtacaaaaaagcaggctcctgcaggaccatg

SEQ ID NO: 20 - DNA, 62 bp, Artificial Sequence
  Synthetic Oligonucleotide
gggggataagcaatgctttcttataatgccaactttgtacaagaaagctgggtctcgagcta

SEQ ID NO: 21 - DNA, 23 bp, Artificial Sequence
  Synthetic Oligonucleotide
gaaactgatgcattgaacttgac

SEQ ID NO: 22 - DNA, 22 bp, Artificial Sequence
  Synthetic Oligonucleotide
gctacattgtttcacaaacttc

SEQ ID NO: 23 - DNA, 1539 bp, Arabidopsis thaliana
atggtcagccatccaatggagaaagctgcaaatggtgcgtctgcgttggaaacgcagacgggtgagttagatcagccggaacggcttcgtaagatcatatcggtgtcttccattgccgccggtgtacagttcggttgggctttacagttatctctgttgactccttacgtgcagctactcggaatcccacataaatgggcttctctgatttggctctgtggtccagtctccggtatgcttgttcagcctatcgtcggttaccacagtgaccgttgcacctcaagattcggccgtcgtcgtcccttcatcgtcgctggagctggtttagtcaccgttgctgttttccttatcggttacgctgccgatataggtcacagcatgggcgatcagcttgacaaaccgccgaaaacgcgagccatagcgatattcgctctcgggttttggattcttgacgtggctaacaacaccttacaaggaccctgcagagctttcttggctgatttatcagcagggaacgctaagaaaacgcgaaccgcaaacgcgtttttctcgtttttcatggcggttggaaacgttttgggttacgctgcgggatcttacagaaatctctacaaagttgtgcctttcacgatgactgagtcatgcgatctctactgcgcaaacctcaaaacgtgttttttcctatccataacgcttctcctcatagtcactttcgtatctctctgttacgtgaaggagaagccatggacgccagagccaacagccgatggaaaagcctccaacgttccgtttttcggaggaatcttcggagctttcaaggaactaaaaagacccatgtggatgcttcttatagtcactgcactaaactggatcgcttggttccctttccttctcttcgacactgattggatgggccgtgaggtgtacggaggaaactcagacgcaaccgcaaccgcagcctctaagaagctttacaacgacggagtcagagctggtgctttggggcttatgcttaacgctattgttcttggtttcatgtctcttggtgttgaatggattggtcggaaattgggaggagctaaaaggctttggggtattgttaacttcatcctcgccatttgcttggccatgacggttgtggttacgaaacaagctgagaatcaccgacgagatcacggcggcgctaaaacaggtccacctggtaacgtcacagctggtgctttaactctcttcgccatcctcggtatcccccaagccattacgtttagcattccttttgcactagcttccatattttcaaccaattccggtgccggccaaggactttccctaggtgttctgaatctagccattgtcgtccctcagatggtaatatctgtgggaggtggaccattcgacgaactattcggtggtggaaacattccagcatttgtgttaggagcgattgcggcagcggtaagtggtgtattggcgttgacggtgttgccttcaccgcctccggatgctcctgccttcaaagctactatgggatttcattga

(19) United States
(12) Patent Application Publication
(10) Pub. No.: US 2004/0115642 A1
(43) Pub. Date: Jun. 17, 2004

(54) Single-step methods for gene cloning

(76) Inventor: Changlin Fu, Chesterfield, MO (US)

Correspondence Address: MONSANTO COMPANY, 800 N. LINDBERGH BLVD., ATTENTION: G.P. WUELLNER, IP PARALEGAL, (E2NA), ST. LOUIS, MO, 63167, US

(21) Appl. No.: 10/318,390

(22) Filed: Dec. 12, 2002

(51) Int. Cl. C12Q 1/68; C12P 19/34; C12N 15/74

(52) U.S. Cl. 435/6; 435/91.2; 435/471

(57) ABSTRACT

The present invention provides compositions and methods for one-step recombinational cloning. The compositions include vectors having multiple recombination sequences with unique specificity. The methods permit the cloning of nucleic acid molecules in a one-step process.

### SEQUENCE LISTING

Two copies of the sequence listing (Seq. Listing Copy 1 and Seq. Listing Copy 2) and a computer-readable form of the sequence listing, all on CD-ROMs, each containing the file named “52920. ST25. ascii”, which is 9,898 bytes (measured in MS-DOS) and was created on Dec. 6, 2002, are herein incorporated by reference.

### BACKGROUND OF THE INVENTION

The present invention provides methods for cloning nucleic acid molecules using recombinational cloning methods and vectors useful in such method.

Many approaches or methods have been developed and used for gene cloning. Examples of these are cloning by restriction enzyme digestion and ligation of compatible ends, T-A cloning directly from PCR product, TOPO-attached unidirectional cloning, and recombination-based cloning.

Recombination-based cloning is one of the most versatile cloning methods available due to its high cloning efficiency and its broad application for cloning a variety of genes regardless of available restriction enzyme. Recombination cloning uses the lambda recombination system to clone genes into vectors that contain recombination sequences for the lambda recombinase machinery. Recombination cloning uses site-specific recombinases, which along with associated proteins in some cases, recognize specific sequences of bases in a nucleic acid molecule and exchange the nucleic acid segments flanking those sequences. The recombinases and associated proteins are collectively referred to as “recombination proteins”. Site-specific recombinases are proteins that are present in many organisms (e.g., viruses and bacteria) and have been characterized as having both endonuclease and ligase properties.

Many of the known site-specific recombinases belong to the integrase family of recombinases including the Integrase/att system from bacteriophage lambda. An example of one application of the Integrase/att system from bacteriophage lambda is the LR cloning reaction as disclosed in U.S. Pat. Nos. 5,888,732 and 6,277,608 B1 and U.S. published patent application 2002/0007051 A1 and International application WO 02/081711 A1 published under the PCT, all of which are incorporated herein by reference. The LR cloning reaction is commercially available as the GATEWAY™ cloning technology (available from Invitrogen Corporation, Carlsbad, Calif.). The LR cloning reaction is catalyzed by the LR Clonase Enzyme mix, which comprises lambda recombination proteins Int, Xis, and the E. coli-encoded protein IHF.

The nucleic acid molecules participating in the recombination reactions contain site-specific recombination sequences. A recombination sequence is a nucleic acid sequence that is recognized and bound by recombination proteins during the initial stages of integration or recombination. Examples of such recombination sequences include the attL and attR recombination sequences. Some recombination-based systems utilize vectors that contain at least two different site-specific recombination sequences based on the bacteriophage lambda system that are mutated from the wild-type (attO) sequences. Each mutated sequence has a unique specificity for its cognate partner att sequence (i.e., its binding partner recombination sequence) of the same type (for example attL1 with attR1) and will not cross-react with recombination sequences of the other mutant type or with the wild-type attO sequence. Different sequence specificities allow directional cloning or linkage of desired molecules thus providing desired orientation of the cloned molecules. Nucleic acid fragments flanked by recombination sequences are cloned by replacing a selectable marker flanked by att sequences on the recipient plasmid molecule, sometimes termed the Destination Vector.

More specifically, the GATEWAY™ cloning Technology employs a two-step cloning reaction that recreates the lambda recombination reactions in vitro through a cocktail of recombination proteins and a set of vectors containing the att sequences they recognize. With reference to FIG. 1 in the first cloning step, known as the “BP reaction”, a PCR product comprising a gene flanked with recombination sequences attB1 and attB2 (25 base pairs each), which were incorporated during PCR, is crossed with a donor vector plasmid comprising attP1 and attP2 sequences (approximately 232 and 233 base pairs each, respectively) using BP Clonase to catalyze recombination resulting in an entry vector with new recombination sequences attL1 and attL2 (99 and 100 base pairs each) flanking the gene. In the second cloning step, known as the “LR reaction”, the entry vector comprising the gene is crossed with a destination vector comprising a selectable marker flanked by attR1 and attR2 sequences (125 and 158 base pairs each) using LR Clonase to catalyze recombination resulting in an expression clone. The expression clone comprises the gene inserted correctly positioned (same orientation and reading frame) in a desirable vector backbone.

What is needed in the art is a cloning method applicable to a variety of gene sequences, having a high cloning efficiency, and requiring only a one-step cloning reaction. This method would enable researchers to decrease the time and cost associated with gene cloning. The present invention provides such one-step cloning methods using recombination-based cloning to join or link multiple nucleic acid molecules into one or more vectors.

### BRIEF SUMMARY OF THE INVENTION

This invention provides a novel cloning method using LR Clonase to recombine a PCR product having recombination sequences on each end of a linear nucleic acid molecule with a destination vector.

This invention also provides a destination vector comprising a promoter adjacent to a recombination sequence and two bacteria lethal genes situated between two recombination sequences.

### BRIEF DESCRIPTION OF THE DRAWINGS

FIG. 1 illustrates a two-step cloning method of the prior art.

FIG. 2 illustrates a one-step cloning method of present invention.

FIGS. 3, 4, and 5 illustrate elements and sites on plasmids useful as destination vectors in the practice of this invention.

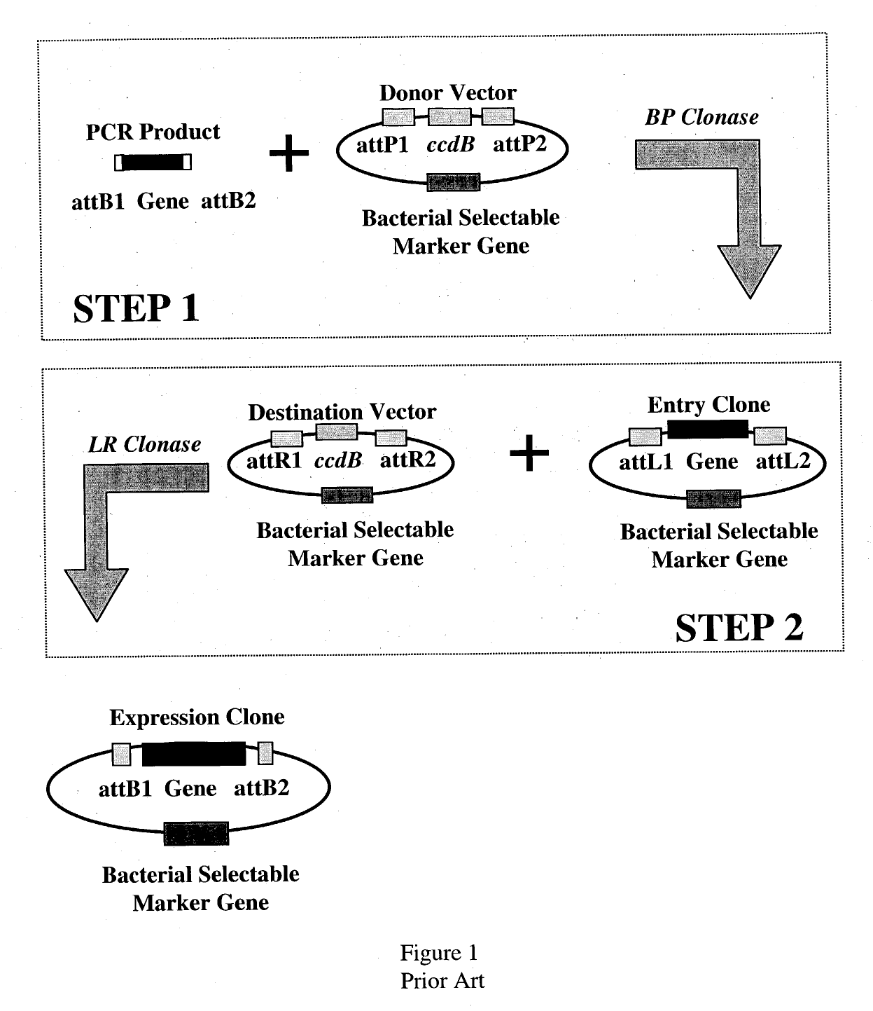

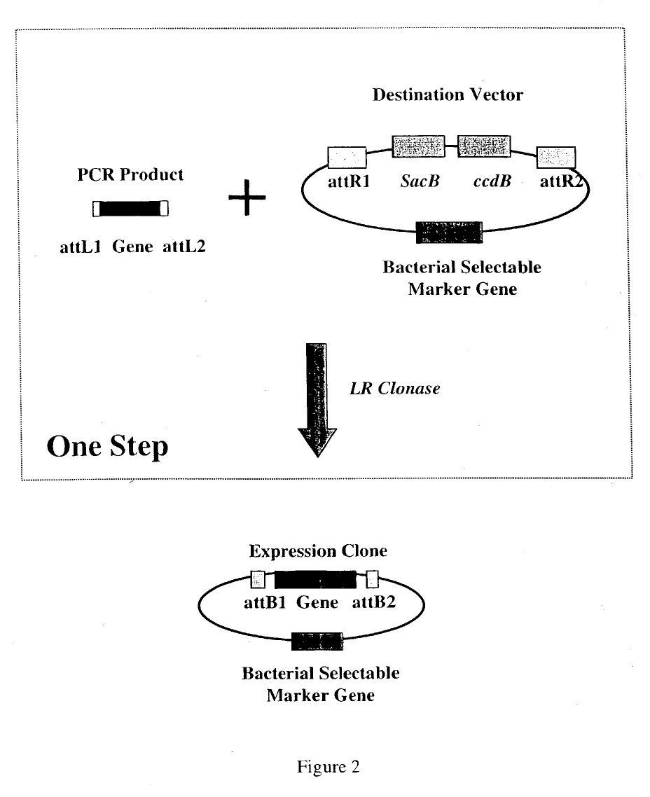

### DETAILED DESCRIPTION OF THE INVENTION

The following detailed description of the invention is provided to aid those skilled in the art to practice the present invention. Even so, the following detailed description should not be construed to unduly limit the present invention as modifications and variations in the embodiments discussed herein may be made by those of ordinary skill in the art without departing from the spirit or scope of the present inventive discovery.

The present invention generally provides materials and methods for joining or combining two molecules of nucleic acid by a one-step recombination reaction between recombination sequences, two of which are present on each nucleic acid molecule. Accordingly, the invention relates to methods for creating novel or unique combinations of nucleic acid molecules.

As used herein, the term “recombination” means a processes based on phage lambda site-specific recombination. DNA recombination sequences and proteins mediate the recombination reactions.

As used herein, the term “cloning” means the transfer of a specific nucleic acid molecule from a first source to a second source.

As used herein, the term “specific nucleic acid molecule” means a nucleic acid molecule to be cloned by the methods of the invention. The specific nucleic acid molecule is used to produce one or more “PCR products” incorporating all or a portion of the specific nucleic acid molecule. In one aspect, the PCR product produced by the methods of the invention comprises two recombination sequences. In a preferred aspect, the PCR product is a specific nucleic acid molecule having a recombination sequence added to each end. In another preferred aspect, when two recombination sequences are added to a specific nucleic acid molecule of interest, such recombination sequences do not substantially recombine with each other. Each recombination sequence on the PCR product has a corresponding binding partner recombination sequence located on the destination vector to be joined by the methods of the invention. For instance, a PCR product to be used in the practice of this invention comprises a first and second recombination sequence added to each end of a linear specific nucleic acid molecule. This PCR product is used with a destination vector that comprises a third and fourth recombination sequence flanking at least one bacterial lethal gene. The first and second recombination sequences preferably do not recombine with each other and the third and fourth recombination sequences preferably do not recombine with each other, although the first and third and/or the second and fourth recombination sequences do recombine.

The specific nucleic acid molecules for use in the present invention can be any nucleic acid molecule derived from any source or produced by any means known to those skilled in the art including, but not limited to, amplification such as by PCR, isolation from natural sources, chemical synthesis, shearing or restriction digest of larger nucleic acid molecules (such as genomic or cDNA), transcription, reverse transcription, and the like. Such molecules may be derived from or comprise any nucleic acid molecule from any natural sources such as cells (e.g., prokaryotic cells or eukaryotic cells), viruses, tissues, organs (such as organs from any animal, plant, or other source), and organisms or may be derived from or comprise any nucleic acid molecule from non-natural or synthetic sources (e.g., derivative nucleic acids). The nucleic acid molecules of the invention may also comprise protein-coding sequences, non-coding sequences that serve expressive functions (e.g., promoter regions, translation termination regions, or other sequences), or non-coding sequences that serve non-expressive functions (e.g., untranslated regions, multiple-coding regions, or other sequences). Recombination sequences may be added to such molecules by any means known to those skilled in the art including amplification or nucleic acid synthesis using primers containing said recombination sequences.

The term “recombinant vector” means any agent such as a plasmid, cosmid, virus, autonomously replicating sequence, phage, or linear or circular single-stranded or double-stranded DNA or RNA nucleic acid molecule, derived from any source, capable of genomic integration or autonomous replication, comprising a DNA molecule where one or more DNA molecules have been linked in a functionally operative manner. Such recombinant DNA vectors may be capable of introducing a 5′ regulatory sequence or promoter region and a DNA molecule for a selected gene product into a cell in such a manner that the DNA molecule is transcribed into a functional mRNA molecule that is translated and therefore expressed as a protein product. Recombinant vectors may be constructed to be capable of expressing antisense RNA molecules, in order to inhibit translation of a specific RNA molecule of interest.

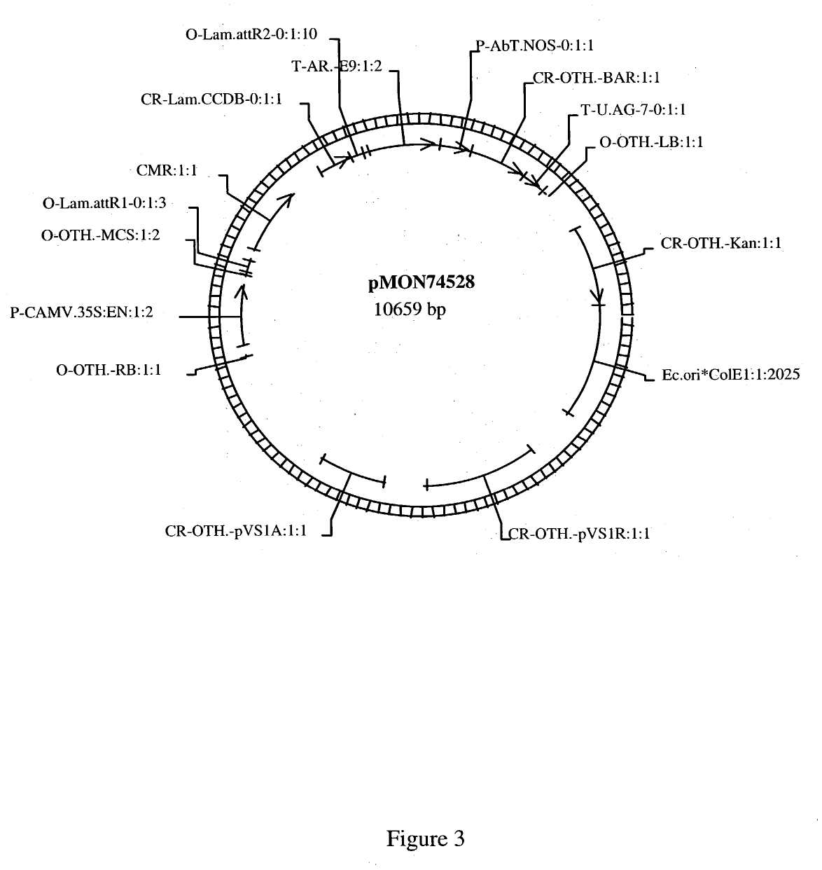

The term “destination vector” as used herein means a recombinant vector comprising two recombination sequences and a promoter for gene expression adjacent to one of the recombination sequences. In a preferred aspect of the invention, the recombination sequences flank at least one selection gene. Plasmids that are useful as destination vectors are shown in FIGS. 3-5.

The destination vector is used to produce the “expression clone”. The expression clone comprises all or a portion of the destination vector and all or a portion of the specific nucleic acid molecule. The expression, clone created by the methods of the invention may be used for any purpose known to those skilled in the art. For example, the expression clones produced by the methods of the invention may be used for plant transformation to produce transgenic plants which express proteins or peptides encoded by the nucleic acid molecule.

As used herein, “LR Clonase” means an enzyme mixture comprising the lambda recombination proteins Int and Xis and the E. coli-encoded protein IHF. In the practice of this invention, the LR Clonase enzyme mix available from Invitrogen Corporation, Carlsbad, Calif. is particularly useful.

### One-Step Cloning Method

This invention provides a method for cloning a specific nucleic acid molecule. This method uses LR Clonase to recombine a PCR product having recombination sequences on each end of a linear nucleic acid molecule with a destination vector.

In one aspect, the method of the present invention may use a single pair of oligonucleotide primers (comprised of a sense primer and an antisense primers) during a single polymerase chain reaction to add two recombination sequences to a specific nucleic acid molecule. Each oligonucleotide primer comprises, in part, a recombination sequence, a gene specific sequence, and optionally a terminus specific sequence comprising a linker sequence to produce a PCR product to be used directly with a destination vector. Given the current state of the art, this is not the preferred method of practicing the invention, but given the remarkable progress being made in molecular biology it is anticipated that this could become the preferred method for practicing the invention.

Thus, in another aspect the method of the present invention may use two pairs of oligonucleotide primers during two polymerase chain reactions to add two recombination sequences to a specific nucleic acid molecule to produce a PCR product to be used directly with a destination vector. Each primer of the first pair of oligonucleotide primers (comprised of a sense primer and an antisense primer) comprises, in part, a gene specific sequence adjacent to a terminus specific sequence itself comprising a linker sequence and an overlapping portion of the recombination sequences. Each primer of the second pair of oligonucleotide primers (comprised of a sense and an antisense primer) comprises, in part, a recombination sequence.

The gene specific sequence is designed to hybridize to a specific terminus of the specific nucleic acid molecule. Preferably, the gene specific sequence comprises 5-30 nucleotides designed to hybridize to a specific terminus of the specific nucleic acid molecule.

The terminus specific sequence is designed to add any desirable sequence between the gene specific sequence and the recombination specific sequence. The terminus specific sequence may include a linker sequence. The linker sequence may comprise a variety of nucleic acid sequences (or combinations thereof) including, but not limited to sequences suitable for use as primer sites (e.g., sequences which a primer such as a sequencing primer or amplification primer may hybridize to initiate nucleic acid synthesis, amplification or sequencing), Kozak sequences, restriction enzyme recognition sequences, start codons, and transcription and/or translation termination signals such as stop codons (which may be optimally suppressed by one or more suppressor tRNA molecules). For use in the two polymerase chain reactions aspect of this invention, the terminus specific sequence comprises an overlapping portion of a recombination specific sequence. This overlapping portion is designed so that the product of the first polymerase chain reaction hybridizes to the second pair of oligonucleotide primers described above. Preferably, the terminus specific sequence comprises a linker sequence comprising at least 5-30 nucleotides and an overlapping portion of at least one of the recombination specific sequences comprising 5-30 nucleotides designed to hybridize to the terminus of one of the oligonucleotide primers of the second pair described herein. More preferably, the first oligonucleotide primer pair comprises a first oligonucleotide primer comprising a gene specific sequence comprising 5-30 nucleotides designed to hybridize to the 5′ terminus of the specific nucleic acid molecule and a terminus specific sequence comprising one of the sequences provided as SEQ ID NO: 11-12 added to the 5′ terminus of the gene specific sense oligonucleotide primer and a second oligonucleotide primer comprising a gene specific sequence comprising 5-30 nucleotides designed to hybridize to the 3′ terminus of the specific nucleic acid molecule and a terminus specific sequence comprising one of the sequences provided as SEQ ID NO: 13-14 and added to the 3′ terminus of the gene specific antisense oligonucleotide primer.

The recombination sequence is designed to add the recombination sequences referred to as the attL1 (sense) and the attL2 (antisense) recombination sequences and provided as SEQ ID NO: 1-2 or fragments of the full length attL1 or attL2 recombination sequences. The full-length attL1 and attL2 recombination sequences are currently difficult to synthesize as oligonucleotide primers due to concerns well known to one skilled in the art and related to the cost, synthesis efficiency, and quality of such large oligonucleotide primers. Shortening the recombination sequences, therefore, allows for production of oligonucleotide primers containing functional recombination sequences but with less expense and having a higher quality. Preferably, the attL recombination sequences are shortened to about 80% of full length to produce the recombination sequences referred to as the attL1-T4 and attL2-T4 recombination sequences and provided as SEQ ID NO: 9-10. More preferably, the attL recombination sequences are shortened to about 70% of full length to produce the recombination sequences referred to as the attL1-T3 and attL2-T3 recombination sequences and provided as SEQ ID NO: 7-8. More preferably, the attL recombination sequences are shortened to about57% of full length to produce the sequences referred to as the attL1-T1 and attL2-T1 recombination sequences and provided as SEQ ID NO: 5-6. More preferably, the attL recombination sequences are shortened to about 45% of full length to produce the sequences referred to as the attL1-T2 and attL2-T2 recombination sequences and provided as SEQ ID NO: 3-4. Cloning efficiency (i.e. efficiency of recombination) with nucleic acid molecules containing these shortened attL recombination sequences was found to be as high as 75-100%.

In one aspect, the method of the present invention uses a single oligonucleotide primer pair comprising two large primers themselves each comprising a recombination sequence, a terminus specific sequence, and optionally a linker sequence. In another aspect, the method of the present invention uses two oligonucleotide primer pairs, where the each of the second oligonucleotide primer pair comprises, in part, the recombination sequence. The full-length attL1 and attL2 recombination sequences provided as SEQ ID NOS: 1-2 have a sequence identity of 95% to each other. High sequence identity between sense and antisense primers is well known to one skilled in the art to be associated with decreased productivity of a polymerase chain reaction, thus making a polymerase chain reaction using these sequences as oligonucleotide primers difficult or even non-productive. The addition of an additional nucleic acid sequence to the recombination sequences increases the production of PCR products. Preferably, the additional nucleic acid comprises the sequence provided as SEQ ID NO: 11-12 which is added to the 3′ terminus of the attL1 recombination sequence or the sequence provided as SEQ ID NO: 13-14 which is added to the 5′ terminus of the attL2 recombination sequence.

The incorporation of recombination sequences by the PCR product allows for the subsequent one-step directional cloning of PCR products into a destination vector. The PCR product is used with a destination vector and LR Clonase to produce the expression clone. The expression clone can be introduced into a host cell of choice for expression of the encoded protein of the specific nucleic acid molecule, or to provide for reduction of expression of the encoded protein of the specific nucleic acid molecule, for example by antisense or co-suppression methods. Potential host cells for the expression clone include both prokaryotic and eukaryotic cells. Of particular interest is the use of the expression clone produced by practicing the methods of the invention for use in plant transformation.

### Destination Vectors

This invention provides destination vectors for use in cloning a specific nucleic acid molecule. The destination vectors comprise a plant promoter adjacent to a recombination sequence and one or more, preferably two, bacteria lethal genes situated between two recombination sequences.

Destination vectors for use in the invention will comprise a plant promoter to direct transcription of the protein-coding region or the antisense sequence of choice. Numerous promoters, which are active in plant cells, have been described in the literature. These include the nopaline synthase (NOS) promoter and octopine synthase (OCS) promoters carried on tumor-inducing plasmids of Agrobacterium tumefaciens or caulimovirus promoters such as the Cauliflower Mosaic Virus (CaMV) 19S or 35S promoter (U.S. Pat. No. 5,352,605), and the Figwort Mosaic Virus (FMV) 35S-promoter (U.S. Pat. No. 5,378,619). These promoters and numerous others have been used to create recombinant vectors for expression in plants. Any promoter known or found to cause transcription of DNA in plant cells can be used in the present invention. Other useful promoters are described, for example, in U.S. Pat. Nos. 5,378,619; 5,391,725; 5,428,147; 5,447,858; 5,608,144; 5,614,399; 5,633,441; and 5,633,435, all of which are incorporated herein by reference.

In addition, promoter enhancers, such as the CaMV 35S enhancer (Kay et al. (1987) Science 236: 1299-1302) or a tissue specific enhancer (Fromm et al. (1989) The Plant Cell 1:977-984), may be used to enhance gene transcription levels. Enhancers often are found 5′ to the start of transcription in a promoter that functions in eukaryotic cells, but can often be inserted in the forward or reverse orientation 5′ or 3′ to the coding sequence. In some instances, these 5′ enhancing elements are introns. Deemed to be particularly useful as enhancers are the 5′ introns of the rice actin 1 and rice actin 2 genes. Examples of other enhancers which could be used in accordance with the invention include elements from octopine synthase genes (Ellis et al. (1987) EMBO Journal 6:3203-3208), the maize alcohol dehydrogenase gene intron 1 (Callis et al. (1987) Genes and Develop. 1:1183-1200), elements from the maize shrunken 1 gene, the sucrose synthase intron (Vasil et al. (1989) Plant Physiol. 91:1575-1579) and the TMV omega element (Gallie et al. (1989) The Plant Cell 1:301-311), and promoters from non-plant eukaryotes (e.g., yeast; Ruden et al. (1988) Proc Natl. Acad. Sci. 85:4262-4266).

Destination vectors for use in the invention may also comprise one or more 5′ non-translated leader sequences, which serve to enhance polypeptide production from the resulting mRNA transcripts. Such sequences may be derived from the promoter selected to express the gene or can be specifically modified to increase translation of the mRNA. Such regions may also be obtained from viral RNAs, from suitable eukaryotic genes, or from a. synthetic gene sequence. For a review of optimizing expression of transgenes, see Koziel et al. (1996) Plant Mol. Biol. 32:393-405).

Destination vectors for use in the invention may also comprise a nucleic acid sequence that acts, in whole or in part, to terminate transcription of the specific nucleic acid molecule. One type of 3′ untranslated sequence that may be used is a 3′ UTR from the nopaline synthase gene (nos 3′) of A. tumefaciens (Bevan et al. (1983) Nucleic Acids Res. 11:369-385). Other 3′ termination regions of interest include those from a gene encoding the small subunit of a ribulose-1,5-bisphosphate carboxylase-oxygenase (rbcS), and more specifically, from a rice rbcS gene (PCT Publication WO 00/70066), the 3UTR for the T7 transcript of A. tumefaciens (Dhaese et al. (1983) EMBO J 2:419-426), the 3′ end of the protease inhibitor I or II genes from potato (An et al. (1989) Plant Cell 1:115-122) or tomato (Pearce et al. (1991) Science 253:895-898), and the 3′ region isolated from Cauliflower Mosaic Virus (Timmermans et al. (1990) J Biotechnol 14:333-344). Alternatively, one also could use a gamma coixin, oleosin 3 or other 3′ UTRs from the genus Coix (PCT Publication WO 99/58659).

Destination vectors for use in the invention may also comprise a selectable marker. Selectable markers may be used to select for plants or plant cells that contain the exogenous genetic material. Examples of such include, but are not limited to, a nptII gene (Potrykus et al. (1985) Mol. Gen. Genet. 199:183-188) which codes for kanamycin resistance and can be selected for using kanamycin, G418, etc.; a bar gene which codes for bialaphos resistance; a mutant EPSP synthase gene (Hinchee et al. (1988) Bio/Technology 6:915-922) which encodes glyphosate resistance; a nitrilase gene which confers resistance to bromoxynil (Stalker et al. (1988) J. Biol. Chem. 263:6310-6314); a mutant acetolactate synthase gene (ALS) which confers imidazolinone or sulphonylurea resistance (European Patent Application 154,204 (Sept. 11, 1985)); and a methotrexate resistant DHFR gene (Thillet et al. (1988) J. Biol. Chem. 263:12500-12508.

Destination vectors for use in the invention may also comprise a screenable marker. Screenable markers may be used to monitor transformation. Exemplary screenable markers include a β-glucuronidase or uidA gene (GUS) which encodes an enzyme for which various chromogenic substrates are known (Jefferson (1987) Plant Mol. Biol. Rep. 5:387-405); Jefferson et al. (1987) EMBO J. 6:3901-3907); an R-locus gene, which encodes a product that regulates the production of anthocyanin pigments (red color) in plant tissues (Dellaporta et al. (1988) Stadler Symposium 11:263-282); Other possible selectable and/or screenable marker genes will be apparent to those of skill in the art.

Destination vectors for use in the invention may also comprise a transit peptide for targeting of a gene target to a plant organelle, particularly to a chloroplast, leucoplast or other plastid organelle (U.S. Pat. Nos. 5,728,925 and 5,510,471).

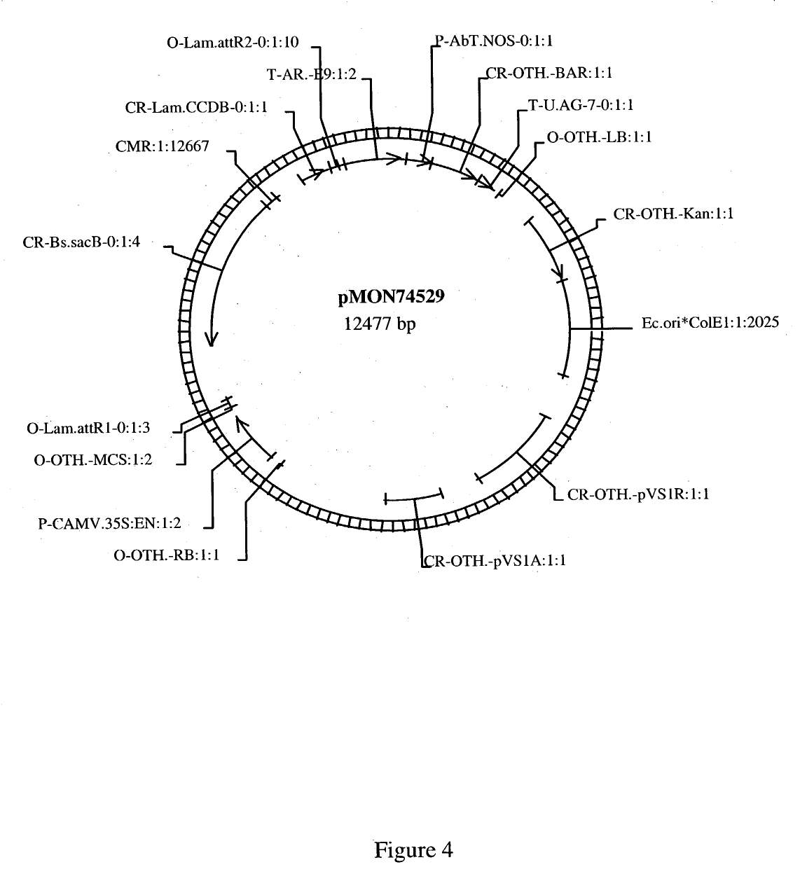

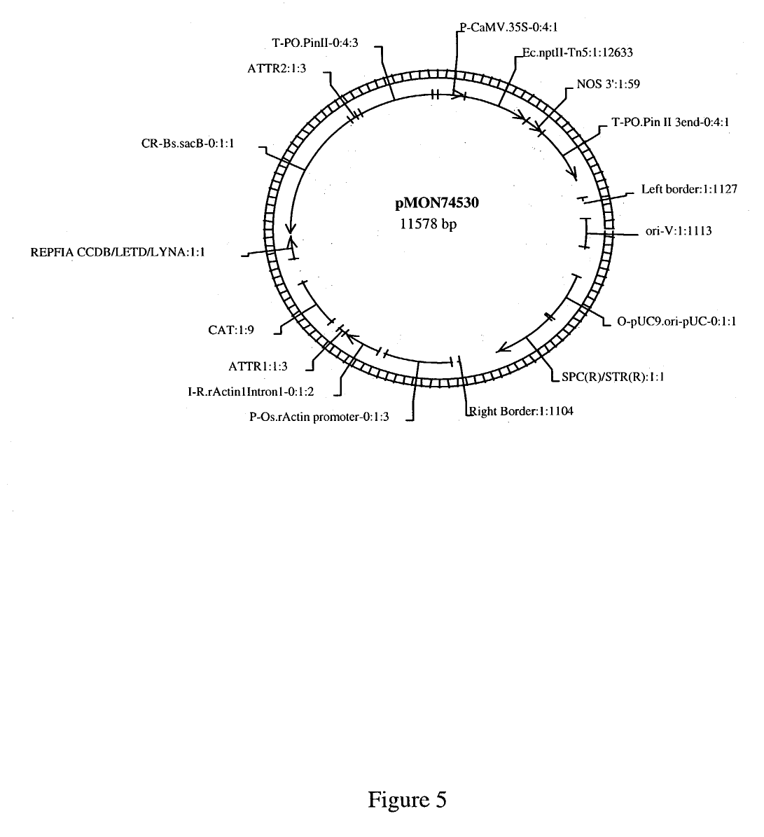

Destination vectors for use in the invention may also be designed to replicate in both E. coli and A. tumefaciens and have all of the features required for transferring large inserts of DNA into plant chromosomes. Design of such vectors is generally within the skill of the art. See, for example, Plant Molecular Biology: A Laboratory Manual, Clark (ed.), Springier, N.Y. (1997). For use in Agrobacterium mediated transformation methods, destination vectors of the invention may also include T-DNA border regions flanking the DNA to be inserted into the plant genome to provide for transfer of the DNA into the plant host chromosome as discussed in more detail below. Exemplary destination vectors that find use in such transformation methods are pMON74528 (FIG. 3), pMON74529 (FIG. 4), and pMON74530 (FIG. 5), which are T-DNA vectors that can be used to clone exogenous genes and transfer them into plants using Agrobacterium-mediated transformation. These destination vectors have the left border and right border sequences necessary for Agrobacterium transformation and the origins of replication for maintaining the plasmid in both E. coli and A. tumefaciens strains. A bacterial selectable marker gene on the destination vector such as the kanamycin resistance gene or spectinomycin/streptomycin resistance gene can be used to select for the presence of the destination vector and subsequent expression clone in E. coli or in A. tumefaciens. These and other similar destination vectors useful for plant transformation may be readily prepared by one skilled in the art. Methods and compositions for transforming bacteria and other microorganisms are known in the art. See for example Sambrook et al. (1989) Molecular Cloning: A Laboratory Manual, Second Edition, Cold Spring Harbor Laboratory Press, Cold Spring Harbor, N.Y.).

The present invention may use negative bacterial selection mechanisms well known to one skilled in the art, where the selection of desirable bacterial colonies (e.g., bacterial colonies containing a vector comprising a desirable insert) may be facilitated by techniques such as expression of a lethal gene-product by undesirable bacterial colonies (e.g. bacterial colonies which do not contain a vector comprising a desirable insert). The selection of desirable bacterial colonies may be made more difficult, however, by false-positive undesirable bacterial colonies (e.g., bacterial colonies containing a vector not comprising a desirable insert). A destination vector designed for double negative selection, one where two different genes are present and each gene encodes a different lethal gene product, may be used in the present invention to decrease the number of surviving false-positive undesirable bacterial colonies, thereby increasing the success rate for selection of desirable bacterial colonies. In one embodiment, double negative selection is practiced with a destination vector comprising both the ccdB gene provided as SEQ ID NO: 16 and the sacB gene and promoter cassette of Bacillus subtilis provided as SEQ ID NO: 15. The ccdB gene product is a potent cytotoxin targeting the essential DNA gyrase of most strains of E. coli, with the notable exception of the DB3.1 E. coli strain. The sacB gene and promoter cassette is sucrose inducible at a sucrose concentration of 7 to 10%. The sacB gene product hydrolyzes sucrose into glucose and fructose and then synthesizes a polymer of fructose, called leven. Accumulation of lethal amount of leven in the periplasm causes cell lysis. Exemplary destination vectors that find use in such a double negative selection mechanism are pMON74529 (FIG. 4) and pMON74530 (FIG. 5).

### Expression Clones

This invention provides methods for producing an expression clone from a PCR product and a destination vector. Such expression clones may be used for a variety of purposes, e.g. to transfer exogenous genetic material into a cell. In plant transformation, exogenous genetic material is transferred into a plant cell. By “exogenous” it is meant that a nucleic acid molecule, for example an expression clone comprising a specific nucleic acid molecule of the present invention, is produced outside the organism, e.g. plant, into which it is introduced. One skilled in the art recognizes that an exogenous nucleic acid molecule can be derived from the same species into which it is introduced or from a different species. Such exogenous genetic material may be transferred into either monocot or dicot plants including, but not limited to, soybean, cotton, canola, maize, wheat, rice, and Arabidopsis plants. Transformed plant cells comprising such exogenous genetic material may be regenerated to produce whole transformed plants.

Technology for introduction of DNA into plant and other eukaryotic cells is well known to those of skill in the art. “Transformation” means a method of introducing a exogenous DNA into a genome and can include any of the well-known and demonstrated methods such as electroporation (as illustrated in U.S. Pat. No. 5,384,253, incorporated herein by reference), microprojectile bombardment (as illustrated in U.S. Pat. Nos. 5,015,580; 5,550,318; 5,538,880; 6,160,208; 6,399,861; and 6,403,865, all of which are incorporated herein by reference), Agrobacterium mediated transformation (as illustrated in U.S. Pat. Nos. 5,635,055; 5,824,877; 5,591,616; 5,981,840; and 6,384,301, all of which are incorporated herein by reference) and protoplast transformation (as illustrated in U.S. Pat. No. 5,508,184, incorporated herein by reference).

After transformation, the transformed plant cells or tissues may be grown in an appropriate medium to promote cell proliferation and regeneration. In the case of protoplasts the cell wall will first be allowed to reform under appropriate osmotic conditions, and the resulting callus introduced into a nutrient regeneration medium to promote the formation of shoots and roots.

It is also to be understood that two different transgenic plants can also be mated to produce offspring that contain two independently segregating added, exogenous genes. Selfing of appropriate progeny can produce plants that are homozygous for both added, exogenous genes that encode a polypeptide of interest. Backcrossing to a parental plant and out-crossing with a non-transgenic plant are also contemplated, as is vegetative propagation.

Expression of a specific nucleic acid molecule is of particular use for production of transgenic plants having improved properties; particularly improved properties that result in crop plant yield improvement.

In addition to the above discussed procedures, those skilled in the art are familiar with the standard resource materials which describe specific conditions and procedures for the construction, manipulation, and isolation of macromolecules (e.g., DNA molecules, plasmids, etc.), as well as the generation of recombinant organisms and the screening and isolating of clones, (see for example, Sambrook et al., Molecular Cloning: A Laboratory Manual, Cold Spring Harbor Press (1989); Mailga et al., Methods in Plant Molecular Biology, Cold Spring Harbor Press (1995; Birren et al., Genome Analysis: Analyzing DNA, 1, Cold Spring Harbor, N.Y. (1998)).

Having now generally described the invention, the same will be more readily understood through reference to the following examples, which are provided by way of illustration, and are not intended to be limiting of the present invention, unless specified.

### EXAMPLE 1

### Destination Vector Construction

This example illustrates the preparation of destination vectors for use in subsequent recombination cloning using prior art technology, i.e. the two attR recombination sequences (from Invitrogen) were cloned into a recombinant vector flanking a chloramphenicol-resistance (Cm^{r}) gene and a ccdB gene. The contiguous attR1, Cm^{r} gene, ccdB gene, and attR2 sequences were moved as a single nucleic acid molecule into a recombinant vector to provide a destination vector of double-stranded DNA plasmid as illustrated in FIG. 3 designated as pMON74528. FIG. 3 illustrates the vector pMON74528 destination vector comprised of the Agrobacterium right border (O-OTH.-RB), e35S promoter (P-CAMV.35S), attR1 (O-Lam.attR1), Chloramphenicol resistance gene (CMR), ccdB gene (CR-Lam.CCDB), attR2 (O-Lam.attR2), Arabidopsis E9 terminator (T-AR.E9), Agrobacterium NOS promoter (P-AbT.NOS), bar gene (CR-OTH.-BAR), AG terminator, Agrobacterium left border (O-OTH.-LB), kanamycin resistance gene (CR-OTH.-Kan), E. coli origin of replication (Ec.ori*ColE), pVSIR replicon for stabilizing vectors (CR-OTH.-pVS1R), and pVS1A replicon for stabilizing vectors (CR-OTH.-pVS1A).

The destination vector illustrated in FIG. 4 designated as pMON74529 was constructed by cloning the sacB gene and promoter cassette into the pMON74528 destination vector between the attR1 sequence and the ccdB gene. The pMON74529 destination vector is a double negative selection vector comprised of both the ccdB gene and sacB gene and native promoter cassette. FIG. 4 illustrates the vector pMON74529 double negative selection destination vector comprised of the Agrobacterium right border (O-OTH.-RB), e35S promoter (P-CAMV.35S), attR1 (O-Lam.attR1), sacB gene (CR-BS.sacB), ccdb gene (CR-Lam.CCDB), attR2 (O-Lam.attR2), Arabidopsis E9 terminator (T-AR.E9), Agrobacterium NOS promoter (P-AbT.NOS), bar gene for bialaphos resistance (CR-OTH.-BAR), AG terminator, Agrobacterium left border (O-OTH.-LB), kanamycin resistance gene (CR-OTH.-Kan), E. coli origin of replication (Ec.ori*CoIE), pVS1R replicon for stabilizing vectors (CR-OTH.-pVS1R), and pVS1A replicon for stabilizing vectors (CR-OTH.-pVS1).

The destination vector illustrated in FIG. 5 designated as pMON74530 was constructed by cloning the sacB gene and promoter cassette into a destination vector between the attR1 sequence and the ccdB gene. The pMON74530 destination vector is a double negative selection vector comprised of both the ccdB gene and sacB gene and promoter cassette. FIG. 5 illustrates the vector pMON74530 double negative selection destination vector comprised of the Agrobacterium right border (Right Border), rice actin promoter (P-Os.rActin promoter), rice actin intron (I-R.rActin1Intron), attR1 (ATTR1), Chloramphenicol resistance gene (CAT), ccdB gene (REPFIA CCDB/LETD/LYNA), sacB gene (CR-BS.sacB), attR2 (ATTR2), Potato proteinase inhibitor II polyadenylation site and transcription terminator (T-PO.PinII), e35S promoter (P-CAMV.35S), neomycin and kanamycin resistance gene (Ec.nptII-Tn5), Agrobacterium NOS terminator (NOS 3′), Potato proteinase inhibitor II 3′ end fragment (T-PO. PinII 3 end), Agrobacterium left border (Left Border), broad host origin of replication (ori-V), origin of replication (O-pUC9.ori-pUC), and the spectinomycin and streptomycin resistance gene (SPC(R)/STR(R)).

The destination vectors were amplified in Library Efficiency® DB3.1™ cells (available from Invitrogen Corporation, Carlsbad Calif.) under chloramphenicol selection (25 μg/ml) and kanamycin selection (50 μg/ml) for pMON74528 and kanamycin selection (50 μg/ml) for pMON74529. Vector DNA was purified from bacterial cultures using a QIAGEN Plasmid Kit (available from QIAGEN Inc. Valencia Calif.).

### EXAMPLE 2

### Primer Design

This example illustrates the design of primers used for cloning the specific nucleic acid molecule for the AtSUC2 coding sequence and the attL recombination sequences.

The sequence of the AtSUC2 gene was used for primer design and is provided as SEQ ID NO: 23. The primers designed as the first pair of oligonucleotide primers comprising the two termini specific nucleotide sequences are provided as SEQ ID NOS: 17-18. The primers are designed as follows:

Sense Primer:
5′-CTCCTGCAGGACCATGGTCAGCCATCCAATGGAGA-3′
Antisense Primer:
5′-CTGGGTCTCGAGCTAATGAAATCCCATAGTAGCTT-3′

where in each-primer the underlined portion of the sequence represents the terminus specific sequence and the portion of the sequence that is not underlined represents the gene specific sequence.

The primers designed as the second pair of oligonucleotide primers comprising the two recombination specific nucleotide sequences are provided as SEQ ID NOS: 19-20. The primers are designed as follows:

Sense Primer attL1-T1:
5′-CCCCGATGAGCAATGCTTTTTTATAATGCCAACTTTGTA
CAAAAAAGCAGGCTCCTGCAGGACCATG-3′
Antisense Primer attL2-T1:
5′-GGGGGATAAGCAATGCTTTCTTATAATGCCAAGTTTGTA
CAAGAAAGCTGGGTCTCGAGCTA-3′

where in each primer the underlined portion of the sequence represents the terminus specific sequence and the portion of the sequence that is not underlined represents the recombination sequence.

### EXAMPLE 3

### Primer Incorporation and PCR of Genes

This example illustrates the production of an amplified DNA molecule comprising a specific nucleic acid molecule bounded by recombination sequences useful for one-step recombination cloning. More specifically, two polymerase chain reactions were performed with the oligonucleotide primers described in Example 2 to produce nucleic acid molecules comprising the AtSUC2 coding sequence and the recombination sequences for subsequent one step recombination cloning.

The first polymerase chain reaction was done to add two termini specific nucleotide sequences to the ends of the AtSUC2 coding sequence molecule. The first polymerase chain reaction amplified the AtSUC2 coding sequence with the first pair of oligonucleotide primers (comprised of a sense and an antisense primer) provided as SEQ ID NO: 17-18 using Herculase™ polymerase (available from Stratagene, La Jolla Calif.).

The second polymerase chain reaction was done to add two recombination specific nucleotide sequences to the ends of the first PCR product. The second polymerase chain reaction amplified the product of the first polymerase chain reaction with the second pair of oligonucleotide primers (comprised of a sense and an antisense primer) provided as SEQ ID NO: 19-20.

The product of the second polymerase chain reaction was loaded on a 0.7% agarose gel and excised with a scalpel and purified using the QIAquick® PCR Purification Kit (available from QIAGEN Inc., Valencia Calif.).

### EXAMPLE 4

### One-Step Cloning Reaction and Double Negative Selection

This example illustrates a one-step recombination cloning reaction according to this invention. The linear purified second PCR product prepared as illustrated in example 3 and the destination vector pMON74529 prepared as illustrated in example 1 were used for a subsequent recombination reaction to produce a final (circular) vector comprised of the attL recombination sequences and the AtSUC2 gene to be used for plant transformation. Each recombination reaction contained 4 μL of Gateway™ LR Clonase™ Reaction buffer (available from Invitrogen Corporation Carlsbad Calif.), approximately 100-300 ng of the second PCR product, approximately 250 ng of destination vector, 4 μL of Gateway™ LR Clonase™ Enzyme Mix (available from Invitrogen Corporation, Carlsbad Calif.), and TE buffer to a final volume of 20 μL. The recombination reaction was incubated at room temperature overnight.

Screening of clones (i.e. the selection of positive clones) in E. coli was done using double negative selection. The destination vector pMON74529 is a double negative selection vector comprised of the ccdB gene and the sacB gene and promoter cassette. Bacterial cells (DH10B) transformed with the product of a one step cloning reaction using pMON74529 as the destination vector are plated onto Luria broth agar plus kanamycin at 50 μg/ml in the presence of 10% sucrose. This double negative selection produces two proteins lethal to E. coli unless the lethal genes are removed from the vector. The lethal genes are removed from the vector by a successful recombination reaction.

Approximately 0.5-1.0 μL of each recombination reaction was mixed with 20 μL of ElectroMAX™ DH10B™ competent cells (available from Invitrogen Corporation, Carlsbad Calif.) on ice and loaded into a MicroPulser 0.2 mm electroporation cuvette (Bio-Rad Laboratories Inc., Hercules Calif.) for electroporation. Cells were electroporated at 1.8 kV using a 165-2100 MicroPulser Electroporator (Bio-Rad Laboratories Inc., Hercules Calif.). Electroporated cells were incubated with 80 μL of SOC medium (Invitrogen, Carlsbad Calif.) at 37° C. for 1 hour. Cells were then plated onto LB agar plates containing kanamycin (50 (g/ml) in the presence of 10% sucrose. Plates were incubated overnight at 37° C. Surviving bacterial colonies were expected to contain the antibiotic resistance gene on the destination vector but not contain the lethal genes.

### EXAMPLE 5

### Selection and Sequencing of Clones

This example illustrates the selection and sequencing of the bacterial colonies for a destination vector containing the inserted gene.

Surviving bacterial colonies were picked from each plate. Each well of a 96-deep well plate containing Luria broth plus kanamycin at 50 μg/ml was inoculated with a single colony and incubated in a 37° C. in a shaker at 250 RPM overnight.

Colonies were screened for gene insertion by cultured-cell polymerase chain reaction as follows with universal primers provided as SEQ ID NO: 21-22 which specifically hybridized to the backbone of the vector. The polymerase chain reaction contained 1 μL of the cell culture, 2.5 μL of Platinum® Taq DNA Polymerase High Fidelity buffer (Invitrogen, Carlsbad Calif.), 1.0 μL of MgSO4 (50 mM), 0.5 μL of dNTPs (10 mM), 0.5 μL of 5′ oligo (10 μM), 0.5 μL of 3′ oligo (10 μM), 0.2 μL of Platinum® Taq DNA Polymerase High Fidelity (5U/μL) (Invitrogen, Carlsbad Calif.), and sterile water to a total volume of 25 μL. The PCR was performed according to the conditions suggested by the Platinum® Taq DNA Polymerase High Fidelity (Invitrogen, Carlsbad Calif.) manufacture manual. The polymerase chain reaction was performed in a PTC-225 DNA Engine Tetrad™ thermal cycler (MJ Research Inc., Waltham Mass.).

Bacterial cultures containing the vector with a gene insertion of the appropriate expected size were grown in Luria broth plus kanamycin at 50 μg/ml. Vector DNA was purified from bacterial cultures using a QIAGEN Plasmid Kit (QIAGEN Inc., Valencia Calif.) according to the manufacture's instruction. Purified DNA was sequenced and analyzed using techniques well known to one skilled in the art.

1. A method for cloning a specific nucleic acid molecule comprising using LR Clonase to recombine a PCR product having recombination sequences on each end of a linear nucleic acid molecule with a destination vector.

2. The method for cloning a specific nucleic acid molecule, according to claim 1 wherein said PCR product is produced by adding attL recombination sequences to each end of a linear nucleic acid molecule as a result of a polymerase chain reaction.

3. The method according to claim 2 wherein an attL recombination sequence has a nucleotide sequence of SEQ ID NO: 3.

4. The method according to claim 2 wherein an attL recombination sequence has a nucleotide sequence of SEQ ID NO: 4.

5. The method according to claim 2 wherein an attL recombination sequence has a nucleotide sequence of SEQ ID NO: 5.

6. The method according to claim 2 wherein an attL recombination sequence has a nucleotide sequence of SEQ ID NO: 6.

7. The method according to claim 2 wherein an attL recombination sequence has a nucleotide sequence of SEQ ID NO: 7.

8. The method according to claim 2 wherein an attL recombination sequence has a nucleotide sequence of SEQ ID NO: 8.

9. The method according to claim 2 wherein an attL recombination sequence has a nucleotide sequence of SEQ ID NO: 9.

10. The method according to claim 2 wherein an attL recombination sequence has a nucleotide sequence of SEQ ID NO: 10.

11. A destination vector comprising a promoter adjacent to a recombination sequence and two bacteria lethal genes situated between two recombination sequences.

12. The destination vector according to claim 11 wherein said two bacteria lethal genes comprise the ccdB gene and the sacB gene and promoter cassette.

13. The destination vector according to claim 11 wherein said recombination sequences are attR recombination sequences.
